(19) United States
(12) Patent Application Publication
(10) Pub. No.: US 2010/0249735 A1
(43) Pub. Date: Sep. 30, 2010

(54) Sanitary enhanced absorption wick device

(76) Inventors: Cecelia E. Ali, Niceville, FL (US); Robin R. Waller, Colorado Springs, CO (US); Anita L. Waller, Colorado Springs, CO (US)

Correspondence Address: Cecelia Ali, 14065 Hwy 20 West, Niceville, FL, 32578, US

(21) Appl. No.: 12/661,562

(22) Filed: Mar. 20, 2010

Related U.S. Application Data
(60) Provisional application No. 61/210,932, filed on Mar. 24, 2009.

(51) Int. Cl. A61F 13/15

(52) U.S. Cl. 604/365; 604/378; 604/385.101

(57) ABSTRACT

A separate entity, or wick, that works in conjunction with the user's sanitary pad of choice to help absorb, direct, channel, and contain blood or fluid directly from the vaginal vestibule area onto the sanitary pad. The small wick device fits comfortably and snugly in the vestibule area, and is attached to the top of the sanitary napkin by means of an adhesive border around the bottom portion of the wick. The wick may also either permanently or temporarily attach to a thin, flexible, appendage strip of absorbent material, which is placed between the buttock cheeks to facilitate absorption of blood or fluid leakage particularly while the user is reclining or sleeping.

### CROSS REFERENCE TO RELATED APPLICATIONS

The present application claims priority to U.S. provisional patent application 61/210,932, filed on Mar. 24, 2009. This reference is hereby incorporated in its entirety.

### FIELD OF INVENTION

This present invention relates to female sanitary napkins.

### BACKGROUND OF INVENTION

Throughout history, there has always been a need for products to absorb menstrual and post-partum blood/fluids. This need has been met with the use of rags, sanitary napkins, pads with adhesive strips and tampons. Although these devices have progressively improved over the years, blood/fluid leakage onto under and outer garments and linens remains a problem. This is particularly true while the person is sleeping on their front or back, or reclining for a long period of time. In spite of new contouring of pads and innovative configurations, no single type of pad has all the features that may be desirable to the wearer, and also prevents leakage problems.

### SUMMARY OF INVENTION

The Sanitary Enhanced Absorption Wick Device is a separate entity that works in conjunction with the wearer's sanitary pad of choice. It helps absorb, direct, channel, and contain blood/fluid directly from the vaginal vestibule area onto the sanitary pad of choice. This small, comfortable wick device fits snugly in the vestibule area. It is attached to the top of the sanitary napkin by means of an adhesive border around the bottom portion of the wick. An exemplary embodiment of this device is the addition of a thin, flexible, appendage strip consisting of some kind of absorbent material, which is integrally part of the outer cover layer of the device; or a separate strip which is permanently attached to the basic wick device during the process of manufacturing. This appendage strip is placed between the buttock cheeks, and facilitates absorption of blood/fluid leakage dribbles particularly while the person is reclining or sleeping.

This device is unique in that it is intended to attach onto the top layer of a sanitary pad; and not to a garment. The device is intended to be an supplementary device which will aid in the effectiveness of blood/fluid absorption while wearing a sanitary pad. The device is constructed of semi-moldable, flexible, absorbent material that can comfortably configure to the vestibule area. It can be manufactured in a variety of sizes. The sanitary enhanced absorption wick device is easily attached to sanitary pads. It will help absorb, channel, and wick blood/fluid away from the vestibule area and onto a pad. The wick device will help minimize fluid leakage onto garments and/or linens, which can be costly, annoying, and embarrassing.

### BRIEF DESCRIPTION OF DRAWINGS

The foregoing features of this invention, as well as the invention itself, may be more fully understood from the following description of the drawings in which:

FIG. 1 is a front/top view of an oval perimeter shaped sanitary enhanced absorption wick device with a (1) raised/mounded middle area and a (2) flat ring border area.

FIG. 2 is a front/top view of a round perimeter shaped sanitary enhanced absorption wick device.

FIG. 3 shows the footprint (not to scale) of a wick device with an oval shaped perimeter.

FIG. 4 shows the footprint (not to scale) of a wick device with a round shaped perimeter.

FIG. 5 is a top view of an oval device (4) on a pad (3).

FIG. 6 is a top view of a round device (6) on a pad (5).

FIG. 7 is a side view of the device that is: (a) small, (b) medium, (c) large, and (d) extra large. Not to scale.

FIG. 8 is a side view of the device that is on a pad which is: (a) small/mini, (b) medium, and (c) maxi pad.

FIG. 9 is a side view of a device (7) on a pad (8).

FIG. 10 is a side view of a device (9), that is positioned directly under the vestibule (10) of the vagina (11). It is attached to a pad (12) that is in proper placement under buttock (13) and well below the tailbone area (14).

FIG. 11 is a side view of an absorbent extension appendage strip.

FIG. 12 is a partial side view of the device in which the top layer cover (15) is extended (approx. 6 inches), to create the absorbent extension appendage strip (16). In this embodiment, the strip in an integral part of the cover.

FIG. 13 is a side view of the top layer of the device to which a separate absorbent extension appendage strip (17) has been superficially/permanently (18) attached along one side of the device. In this embodiment, the strip is not formed out of an extension of the cover layer. Instead, it is a separate, yet permanently and securely attached strip incorporated into/onto the top layer of the device; and is part of the construction/manufacture of this embodiment of the device.

FIG. 14 is a side view of the device (19) which has and absorbent extension appendage strip (20), and the device is attached to a pad (21).

FIG. 15 is a side view of the device (22) which is positioned directly under the vestibule area (23) of the vagina (24). The appendage strip (25) is between the buttock cheeks (26), extending toward the tailbone (27) area. The device is attached to a pad (28).

FIG. 16 is a side view of the device (29) that has an absorbent extension appendage strip (30) wrapped around it.

FIG. 17 is a side view of a device in which the absorbent extension appendage strip is partially unwrapped.

FIG. 18 is a side view of the device in which the absorbent extension appendage strip is fully unwrapped and fully extended.

FIG. 19 This is a top view exemplary embodiment of an oval perimeter device of which the top edge/ridge (31) has some texture, and the inner columns/grooves (32), and outer layer columns/grooves (33) are smooth. The center core (34) may be hollow, or filled with absorbent material. The outer ring/border (35) area is smooth and flat.

FIG. 20 Top view of a round perimeter device.

FIG. 21 This is a bottom view of an oval perimeter device showing the inner core area (36). The inner columns are linear and smooth. There is a thin film cover (38) over the adhesive border area which may or may not be extended to the outer edge of the surface layer cover (39).

FIG. 22 is a bottom view of a round perimeter device.

FIG. 23 is a bottom view of an oval perimeter device in which the thin cover film (40) ring has been partially peeled off of the adhesive border (41) of the device.

FIG. 24 is a bottom view of a round perimeter device in which the thin cover film ring is partially peeled off of the adhesive border of the device.

FIG. 25 is a side view of the basic wick device.

FIG. 26 is a partial cut away side view of a device. The outer edge of the adhesive ring (42) is bordered by the inner edge of the outer surface layer cover (43).

FIG. 27 is a side view cross section (enlarged) of the mound/raised center area of the device. The depictions of various layering configurations are for example only. They do not limit, nor change the scope of the basic conceptual idea of the wick. The adhesive area (44) attaches the device to a pad surface (45). The top surface of the device may be slightly textured (46) and has a smooth outer layer covering (47). The inner columns/grooves/channels (48) are tightly bundled. The core (49) area may be hollow or have absorbent material. The device has multiple layers of absorbent material (50).

FIG. 28 is an enlarged bottom view of a round perimeter device. The core (51) has absorbent material in this embodiment. There is a honeycomb of various spongy celled absorbent material in the mound area (52). Linear columns/grooves (53) line the wall of the mound. There is an adhesive border ring (54) around the device, which is flat. There is a thin outer layer cover over the device (55).

FIG. 29 is a top view of an oval perimeter device which has an absorbent extension appendage strip (56).

FIG. 30 is a top view of a round perimeter device which has an appendage strip.

FIG. 31 is a bottom view of an oval perimeter device which has an appendage strip.

FIG. 32 is a bottom view of a round perimeter device which has an absorbent extension appendage strip (57).

FIG. 33 is a top view of the footprint of a device which has a round and: (a) smooth, (b) scalloped, and (c) zig-zag edged perimeter.

FIG. 34 is an embodiment of the device encased in an individual wrapper package.

FIG. 35 is a wrapper/package that has been embellished and/or colored.

FIG. 36 is an example of multiple device packaging.

### DETAILED DESCRIPTION OF THE INVENTION

Sanitary Enhanced Absorption Wick Device that can absorb, direct, and wick blood/fluid from a female person's vaginal vestibule area onto a sanitary pad of choice. It is important to note that the device is a separate entity from the pad, but works in conjunction with a pad. The device is small enough to fit comfortably in the vestibule area. It is constructed of substance that would cause minimal irritation to the genital area. An exemplary embodiment of the device includes an absorbent extension appendage strip that is approximately six inches long (but can vary from one inch to nine inches), which is placed between the buttock cheeks along the curvature of the tailbone area. This extension is particularly helpful when the person is reclining for long periods of time, or sleeping. It helps capture, or wick up the fluid/blood which sometimes dribbles between the buttock cheeks. The device, either with or without the appendage strip, helps absorb blood/fluid by getting closer to the initial source of flow—the vestibule area, and directing/channeling the blood/fluid onto a pad. The device would preferably be constructed of bio-degradable, material substance of renewable source. It is intended for one time use. The device attaches to a pad, not a garment, via a non-toxic adhesive border around the bottom of the device. Because of the small size, and the nature of construction; it is not intended to be used without a pad of some sort. This device does not enter the vaginal area the way modern tampons do. The illustrations and lists of the Exemplary Embodiment pages are to help convey the basic concept of this invention and to help one visualize some of the variations and configurations that are within the scope of the idea. These embodiments are not intended to limit the scope or spirit of the idea in any way. Illustrations are not to scale.

In spite of numerous sizes, shapes, styles, construction material, contour designs and configurations of current sanitary napkins; leakage—especially at night, remains a problem. For those females who do not wish to use tampons, i.e., adolescents, people with heavy periods, post partum women; the sanitary enhanced absorption wick device may provide an extra degree of protection against leakage problems.

The device will work with most pads to help increase the level of protection that many females need. In addition, this product can be economically made, and marketed. This device can be a simple, comfortable, sensible, addition to the sanitary products line.

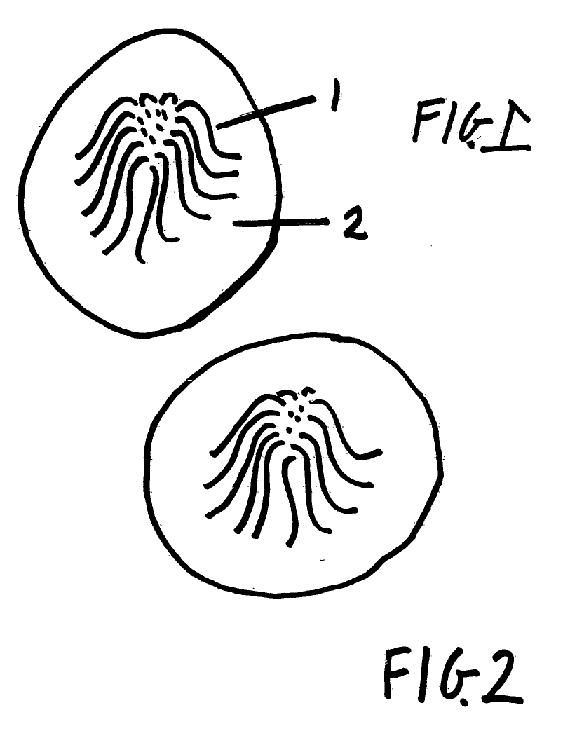

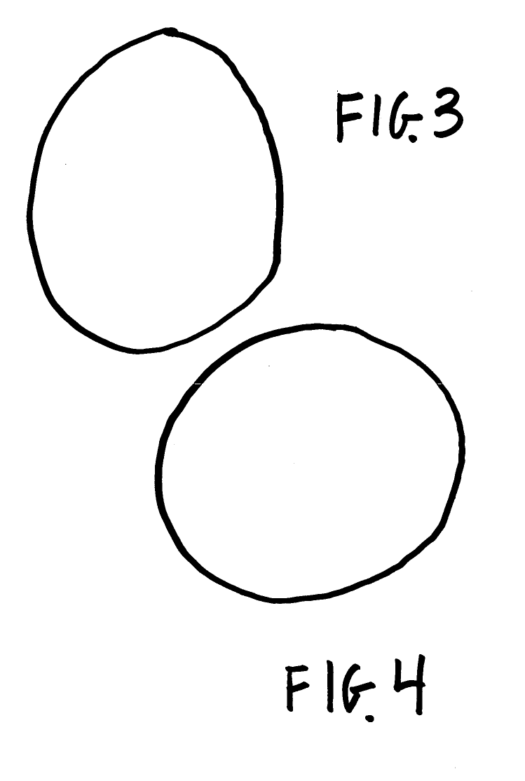

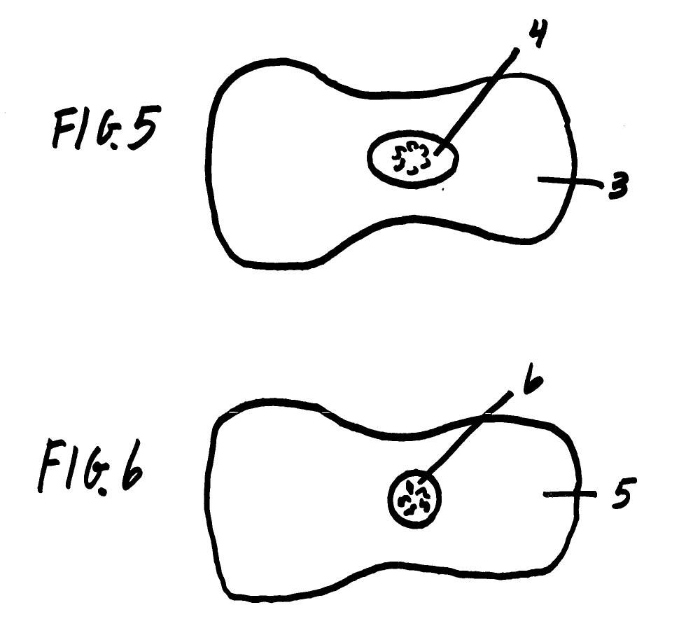

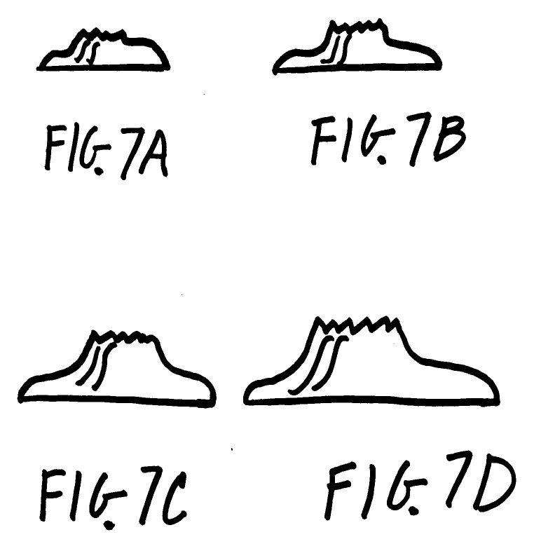

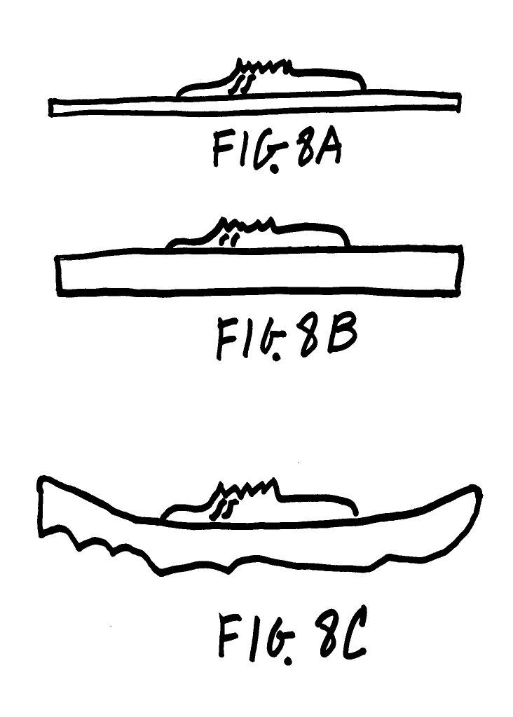

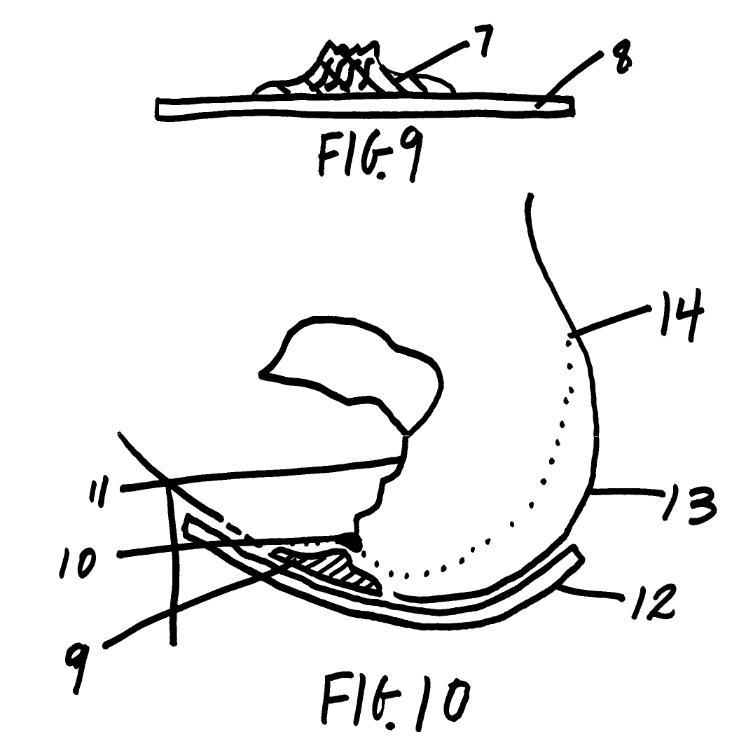

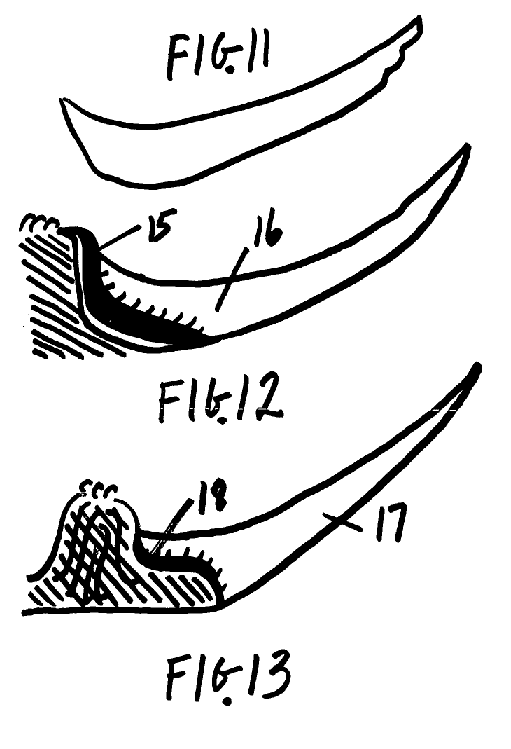

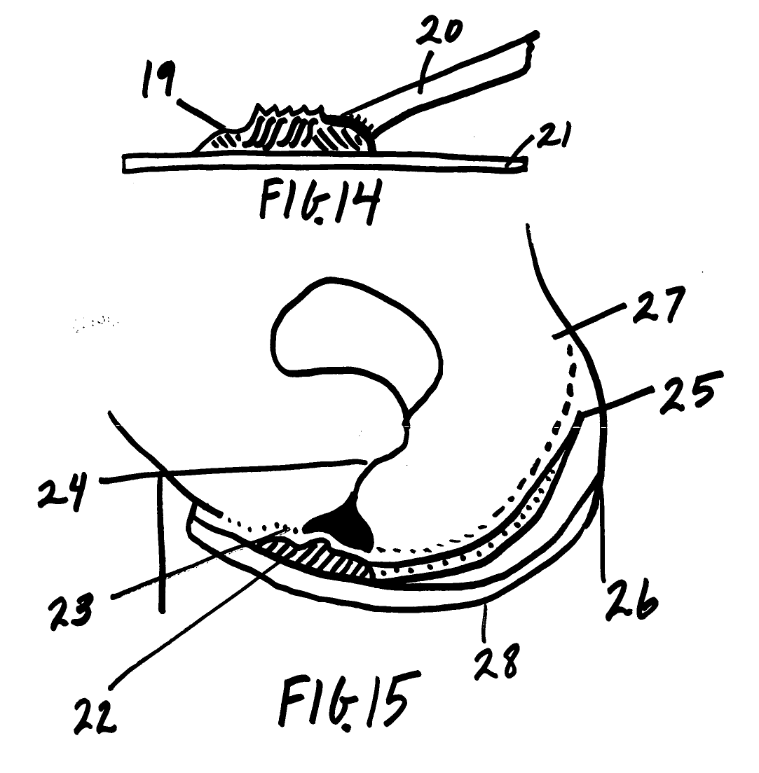

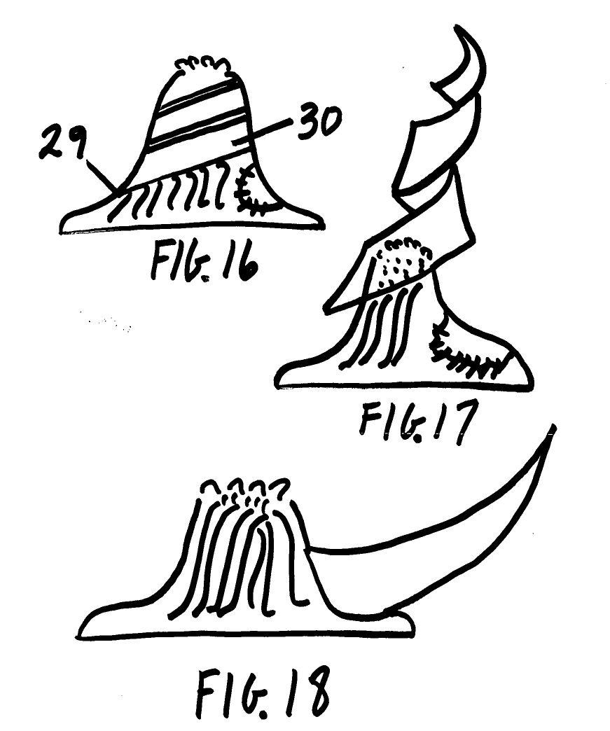

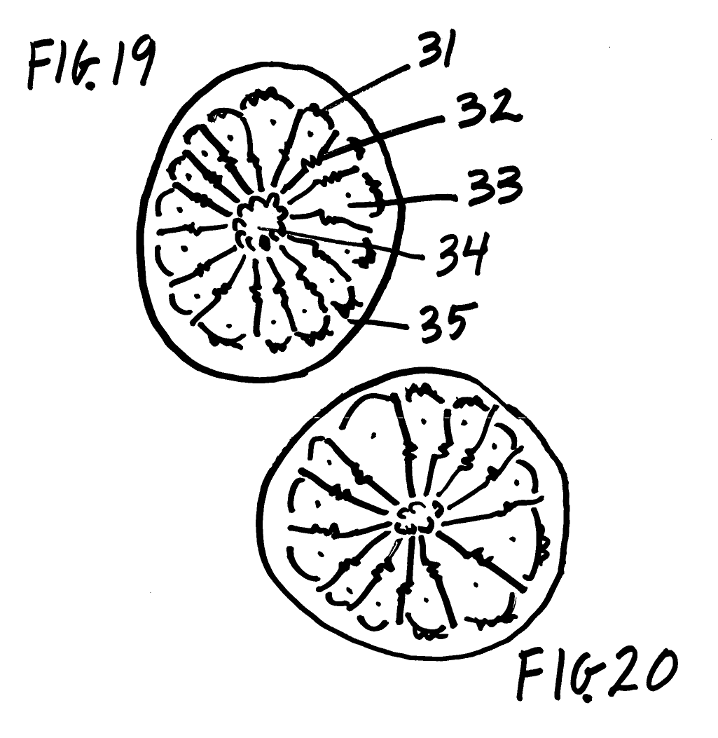

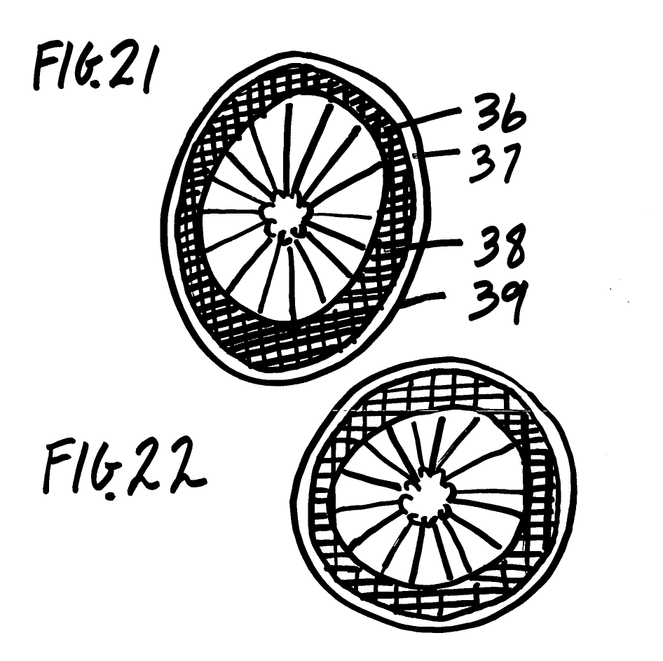

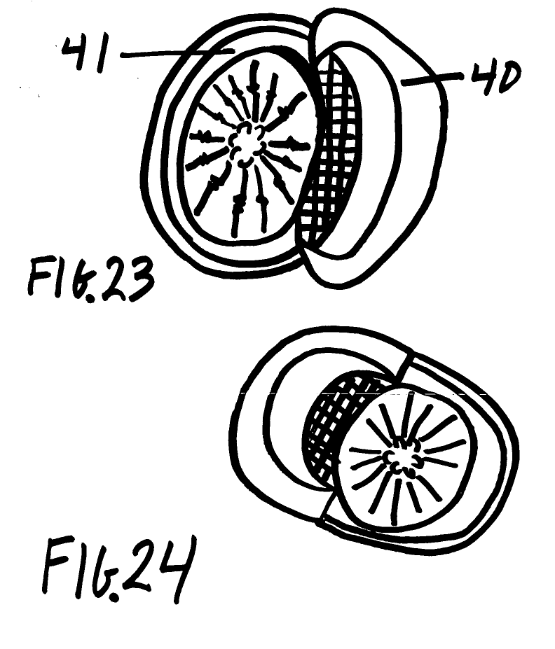

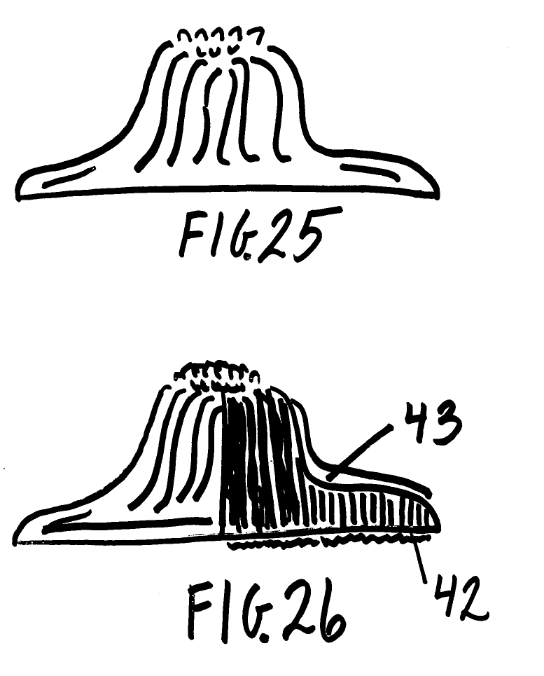

The sanitary enhanced absorption wick device (FIG. 1) and (FIG. 2), has a raised/molded soft, flexible, absorbent middle configuration (1), which conforms to a female person's vulva/vaginal vestibule area. The outer border area of the device (2) is smooth and flat. The device is small/compact—approximately 0.5 to 1.5 inches long, and 0.50 to 1.0 inches wide, and 0.20 to 1.0 inches high. These figures are merely approximations, as there is such a wide range of body sizes in the general population. As such, the device will be made in a variety of sizes to accommodate most girls and women. An Exemplary embodiment of the footprint of the perimeter of the device is either oval (FIG. 3) and/or round (FIG. 4). The device is to be attached/stuck to the top layer of a sanitary napkin of choice. (FIG. 5) is a top view of a pad (3) with and oval perimeter device (4) on it. (FIG. 6) is a top view of a pad (5) with a round perimeter device (6) on it. As seen in (FIG. 7), the device will range in size from (a) small, to (b) medium, to (c) large, and (d) extra large.

Furthermore, the device can be used in conjunction with pads of various sizes or thicknesses. (FIG. 8) shows a device on a: (a) thin mini pad, (b) regular pad, and (c) maxi pad.

The sanitary enhanced absorption wick device (7) as seen in (FIG. 9), is attached to the top middle surface of a pad (8). In (FIG. 10), the wick, and pad are placed between the wearer's legs as usual. The raised mound portion (9), fits snugly at the vestibule (10) opening of the vagina (11). The pad (12) extends normally in relation to the buttock cheeks (13) and the tailbone (14). The wick helps to absorb and redirect blood flow. However, in an alternative embodiment, the wick may be constructed so that the blood flows through it, onto the pad. The adhesive at the bottom of the wick would have to allow the blood to pass through the material while remaining stuck to the top of the sanitary pad.

An Exemplary Embodiment of the device in (FIG. 11), is of an absorbent extension appendage strip that is thin, flexible, and tapered, and approximately six inches long. This extension is placed between the buttock cheeks to act as an additional wicking component of the wick device. The strip (16) can be integrally part of the outer layer covering (15) of the device as seen in (FIG. 12). A section of the top layer covering is purposefully longer and extended on one side of the wick. In another embodiment, (FIG. 13) illustrates how a separately manufactured absorbent extension appendage strip (17) could be applied/incorporated into/onto the outer layer covering as a separate step in the process of construction of the wick. In (FIG. 14), the device (19), which has an appendage strip (20), is attached to a pad (20). The wick may be used alone, and it can also be used with the wick appendage strip.

(FIG. 15) illustrates how the raised mound area of the device (22) fits snugly in the vestibule (23) area of the vagina (24) and the absorbent extension appendage strip (25) is situated between the buttock cheeks (26), following along the curvature of the tailbone (27). The pad (28) is situated under the buttocks as usual. The Exemplary Embodiment depicted in (FIG. 16) shows the basic wick device (29) that has the absorbent extension appendage strip (30) wrapped around it. In (FIG. 17), the appendage strip has been partially unwrapped. In (FIG. 18), the extension appendage strip has been fully unwrapped and extended to its longest length. The wrapped and unwrapped features are not required, however, they assist in compactly manufacturing and packaging the product.

FIG. 19), oval perimeter, and (FIG. 20), round perimeter, show a top view of the device. The top ridge (31) area should be of particularly soft substance material that may have extra texture to increase surface area for better absorption. The inner (32) and outer walls (33) are in a linear/column and/or grooved/channeled configuration to aid in channeling the blood/fluid in a downward direction onto the pad. The core (34) area may be hollow, or filled with absorbent substance material. The entire raised mounded area is soft, pliable, have tensile property, cushioned, spongy and absorbent. It should easily conform to the wearer's vulva/vestibule area, but maintain enough rigidity to fit and stay snugly in said area, even when wet. The border area (35) is smooth and flat.

(FIG. 21), oval perimeter, and (FIG. 22), is a bottom view of the device. The core (36) and inner column walls (37) are smooth and linear. There is a thin, smooth peel off cover film (38) over the adhesive ring portion that borders the outer edge area of the device. The adhesive and/or cover film may, or may not extend to the outer surface layer cover edge (39). In (FIG. 23), oval perimeter device, and (FIG. 24), round perimeter device; the thin, peel-off cover film (40) is partially removed to expose the sticky adhesive ring (41) portion that borders each device. Note that the adhesive does not cover the center bottom portion of the device in this Exemplary Embodiment. However, the center area, as well as the border area of the bottom of the device could also be covered completely with an adhesive layer. In such case, the blood/fluid might be prevented/hindered from flowing onto the center portion of the pad, and possibly decreasing the effectiveness of proper wick/pad absorption. However, this problem might be somewhat lessened and/or eliminated if a lattice type adhesive bottom layer were applied over the entire bottom surface area instead of a solid layer. In such case, the adhesive lattice substance material would have to be able to withstand constant wetness, and still be able to adhere securely to the pad. In any case, the basic premise or concept of this invention is that a slightly raised/mounded absorption wick device is placed, adhered, or stuck to the top middle surface of a pad; and then situated in the vaginal vestibule area. The device may or may not also have an absorbent extension appendage strip attached to it. Then the sanitary napkin pad is affixed to the wearer's garment.

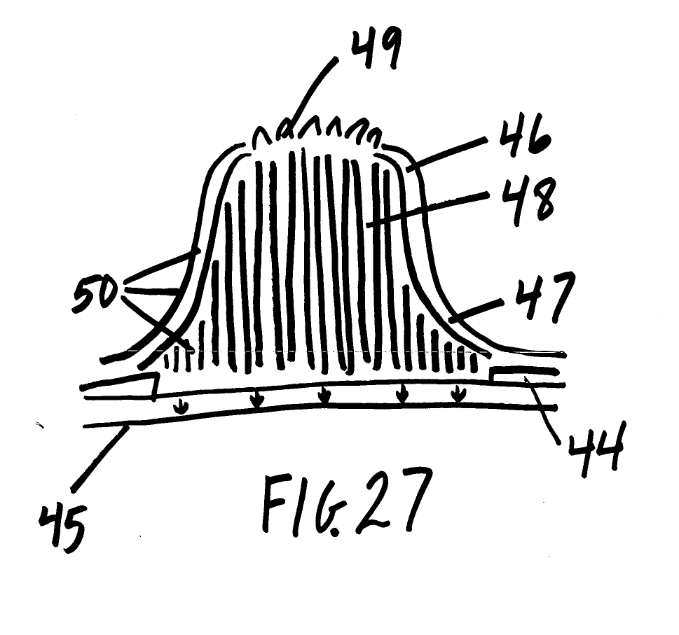

(FIG. 25) is a front/side view of a basic wick device. (FIG. 26) is a partial cross section front/side view of a basic device. The adhesive ring border (42) on the bottom of the device is below, and adjacent to the outer layer cover (43) of the device. The adhesive ring border may, but does not have to, extend to the farthest perimeter edge of the device. (FIG. 27) is an enlarged cross-section side view of the raised mounded portion of the device. The adhesive ring border (44) is attached to the top of a pad (45). Initially, blood/fluid enters through the top (46) and outer surface cover (47) of the device. Blood/fluid flow is directed downward, through the linear, grooved honeycomb of chambers/columns (48). The core (49) area may be hollow, or be filled with absorbent material also. There are several layers (50) of absorbent material substance.

A magnified bottom view of the device in (FIG. 28), shows the core area (51), a honeycomb of absorbent material, (52), linear grooves/columns on the inside walls (53), and adhesive ringed border (54), and the outer layer cover (55). There are a variety of possible material substances which could be used in the manufacture of the device. Some of these might include: cotton, cellulose products, paper pulp, shredded bamboo fiber, rubber, sponge, synthetic material. Preferably, material of a bio-degradable nature, from a renewable source would be chosen. The material would naturally need to be non toxic and hypo-allergenic. An anti-bacterial quality would also be desirable. Regardless of the material used; the device should be moldable, flexible, soft, absorbent, have tensile properties, and be able to maintain the raised, mound shape even when saturated. Either the material used, and/or the way in which it was arranged within the device would need to promote the wicking effect of drawing the blood/fluid from the vestibule area, and onto the pad. The ideal condition would be to keep the top layer of the basic device that is in contact with the skin, as free from wetness as possible.

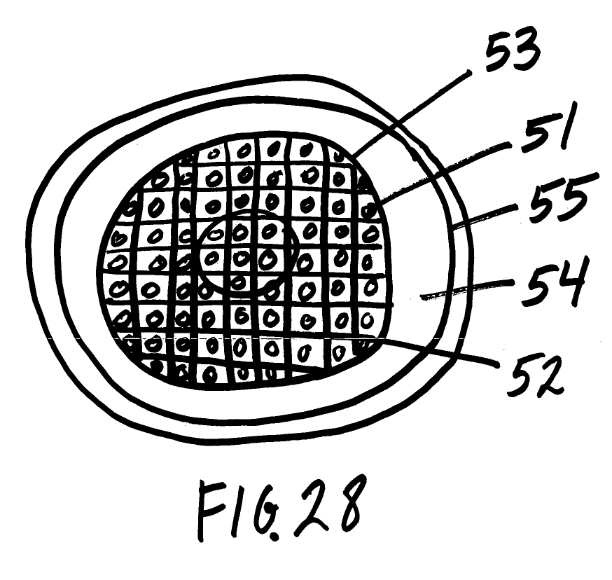

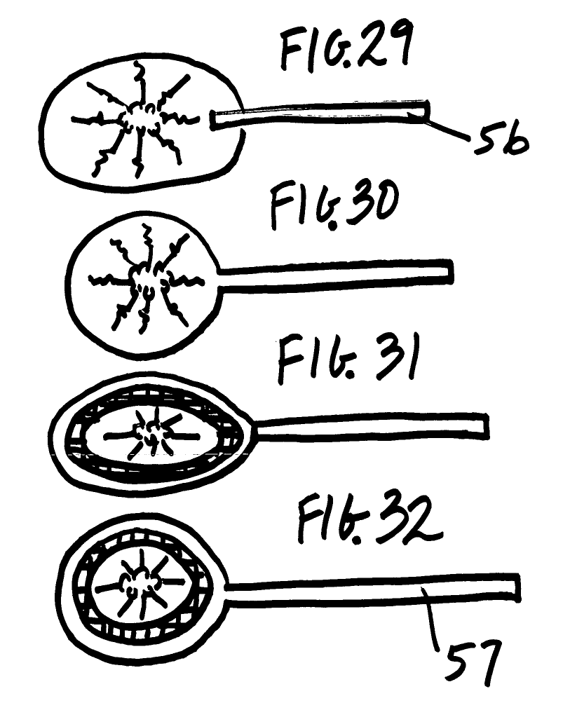

(FIG. 29) oval perimeter, and (FIG. 30), round perimeter, are top views of the device with the exemplary embodiment of the absorbent extension appendage strip (56). (FIG. 31), oval perimeter, and (FIG. 32), round perimeter are bottom views of the device with the exemplary embodiment of the absorbent extension appendage strip (57). The strips are primarily used to capture/catch the blood/fluid that dribbles between the buttock cheeks when in a prolonged reclining position, or while sleeping. Therefore, the strip should have enough absorbency to hold the blood/fluid within the confines of the buttock crevice. In addition, the strip needs to be thin enough so that it is not uncomfortable when worn, and gradually tapers toward the outer end. Also, any portion of the end tip of the strip that extends beyond the buttock crevice should be treated/conditioned/coated so that it does not absorb the blood/fluid. Since the very end tip of the strip may, in some cases, protrude enough to come in contact with a garment; it should not be able to absorb fluid which might come in contact with a garment and soil the garment or linens. In addition, providing a wet-proof end tip will help make removal of the strip from between the buttocks less cumbersome and unpleasant, and more sanitary.

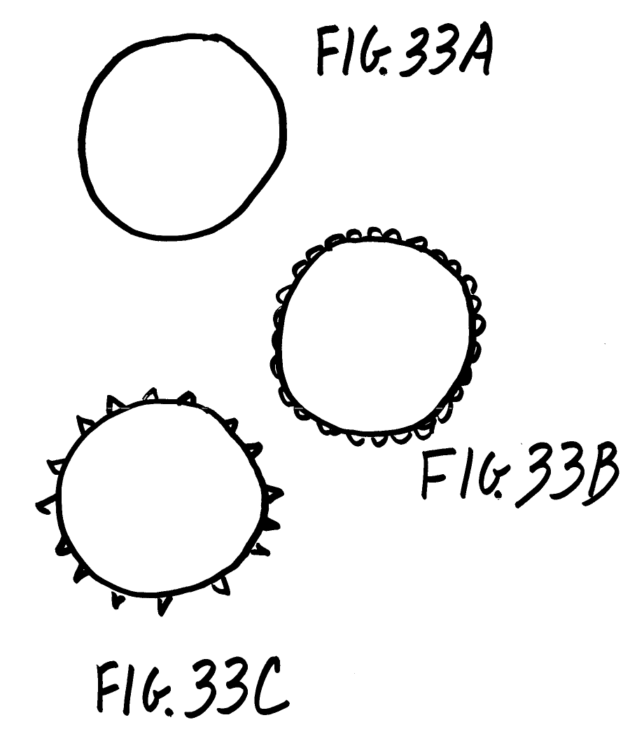

(FIG. 33) is the footprint of a round basic device with: (a) a smooth perimeter edge, (b) scalloped edge, and (c) zig-zagged edge. Variations in edges, or colors other than white, may add interest to the device, and increase marketability.

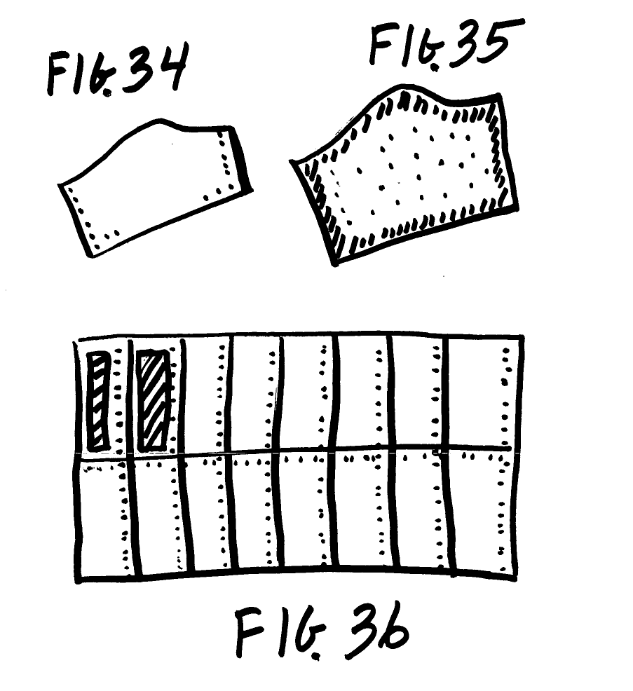

In addition, individually wrapped packaging for each device, (FIG. 34), might make it more portable. The wrappers could also be colored or embellished and decorated (FIG. 35). Devices could be packaged in bulk amounts (FIG. 36) for more affordability.

In summary, the sanitary enhanced absorption wick device, for use in conjunction with a sanitary napkin/pad; will come closer to the source of blood/fluid flow from the vaginal vestibule area. The device will then act as an intermediary component to absorb, capture, and wick the fluids away from the outer surface of the device that come in contact with the genital area, and deposit them into a sanitary pad of choice. The device is held firmly on the middle surface of the pad by means of strong, non-toxic, hypo-allergenic adhesive on the bottom surface of the device. The device which has the absorbent extension appendage strip will be particularly helpful in capturing the blood/fluids that sometimes dribble between the buttock cheeks crevice while reclining for long periods, or sleeping. Using the device will help reduce or eliminate unsightly, costly, and embarrassing leakage stains on garments and linens. In addition, less time may be used to clean up the mess left by these leakage stains. The Sanitary Enhanced Absorption Wick Device and various embodiments will provide a simple, cost effective, and convenient solution to these sanitary pad leakage problems.

We claim:

1. A device for absorbing, directing, channeling, and containing blood or body fluid in conjunction with the user's sanitary pad of choice, comprising:
A soft, modified cone raised configuration, that is moldable to the outside of a female's vaginal vestibule area, wherein
the bottom portion of the device attaches to the top layer of a sanitary pad or napkin using an adhesive, wherein
the device channels the bodily fluid in a downward direction from the user onto the sanitary pad;
bodily fluid flows through the top layer of the wick device onto the pad, and
the device only covers the surface area of the pad that is directly in contact with the user's vestibule area;
an appendage strip or tail is attachable to the wick device and is secured between the buttock cheeks;
the device is disposable.

2. The device of claim 1, wherein the appendage strip is temporarily attached to the wick device.

3. The device of claim 1, wherein the appendage strip is permanently attached to the wick device.

4. A device for absorbing, directing, channeling, and containing blood or body fluid in conjunction with the user's sanitary pad of choice, comprising:
A soft, modified cone raised configuration, that is moldable to the outside of a female's vaginal vestibule area, wherein
the bottom portion of the device attaches to the top layer of a sanitary pad or napkin using an adhesive, wherein
the device channels the bodily fluid in a downward direction from the user onto the sanitary pad;
the device only covers the surface area of the pad that is directly in contact with the user's vestibule area;
bodily fluid is absorbed by the lower layers of the wick device itself;
an appendage strip or tail is attachable to the wick device and is secured between the buttock cheeks;
the device is disposable.

5. The device of claim 4, wherein the appendage strip is temporarily attached to the wick device.

6. The device of claim 4, wherein the appendage strip is permanently attached to the wick device.

7. A device for absorbing, directing, channeling, and containing blood or body fluid in conjunction with the user's sanitary pad of choice, comprising:
A soft, modified cone raised configuration, that is moldable to the outside of a female's vaginal vestibule area, wherein
the bottom portion of the device attaches to the top layer of a sanitary pad or napkin, wherein
the device channels the bodily fluid in a downward direction from the user onto the sanitary pad;
the device only covers the surface area of the pad that is directly in contact with the user's vestibule area.

8. The device of claim 7, wherein the device attaches to the sanitary pad using an adhesive.

9. The device of claim 7, wherein bodily fluid flows through the top layer of the wick device onto the pad.

10. The device of claim 7, wherein bodily fluid is absorbed by the lower layers of the wick device itself.

11. The device of claim 7, wherein an appendage strip or tail is attachable to the wick device and is secured between the buttock cheeks.

12. The device of claim 11, wherein the appendage strip is temporarily attached to the wick device.

13. The device of claim 11, wherein the appendage strip is permanently attached to the wick device.

14. The device of claim 7, wherein the device is disposable.

15. The device of claim 7, wherein the device footprint is oval shaped.

16. The device of claim 7, wherein the device footprint is circular shaped.
